SEQUENCE LISTING (7 sequences)

SEQ ID NO: 1 - protein, 9 aa, Artificial Sequence
  IL-4 receptor targeting peptide
CRKRLDRNC

SEQ ID NO: 2 - protein, 14 aa, Artificial Sequence
  Pro-apoptotic peptide
KLAKLAKKLAKLAK

SEQ ID NO: 3 - protein, 26 aa, Artificial Sequence
  Fusion peptide
CRKRLDRNCGGGKLAKLAKKLAKLAK

SEQ ID NO: 4 - protein, 19 aa, Artificial Sequence
  synthetic sequence
KGGGQVGRQLAIIGDDINR

SEQ ID NO: 5 - protein, 25 aa, Artificial Sequence
  synthetic sequence
LQHRAEVQIARKLQCIADQFHRLHT

SEQ ID NO: 6 - protein, 15 aa, Artificial Sequence
  synthetic sequence
YGRELRRMSDEFVDS

SEQ ID NO: 7 - protein, 7 aa, Artificial Sequence
  synthetic sequence
NSSSVDK

(19) United States
(12) Patent Application Publication
(10) Pub. No.: US 2018/0201651 A1
(43) Pub. Date: Jul. 19, 2018

(54) PHARMACEUTICAL COMPOSITION THAT IS ANTICANCER AND SUPPRESSES CANCER METASTASIS, CONTAINING, AS ACTIVE INGREDIENT, FUSION PEPTIDE SIMULTANEOUSLY TARGETING CANCER CELL AND TUMOR ASSOCIATED MACROPHAGE

(71) Applicant: Kyungpook National University Industry-Academic Cooperation Foundation, Daegu (KR)

(72) Inventors: Byung-Heon Lee, Daegu (KR); Sri Murugan Poongkavithai Vadevoo, Daegu (KR)

(21) Appl. No.: 15/856,740

(22) Filed: Dec. 28, 2017

Related U.S. Application Data
(63) Continuation of application No. PCT/KR2015/012162, filed on Nov. 12, 2015.

(30) Foreign Application Priority Data
Jun. 30, 2015 (KR) 10-2015-0093576

(51) Int. Cl. C07K 7/08; C07K 7/06; A61K 38/10; A61K 38/08; A61K 31/337; A61P 35/00

(52) U.S. Cl. CPC C07K 7/08; C07K 7/06; A61K 38/10; C07K 2319/74; A61K 31/337; A61P 35/00; C07K 2319/33; A61K 38/08

(57) ABSTRACT

The present invention relates to a pharmaceutical composition that is anticancer and suppresses cancer metastasis, containing a fusion peptide as an active ingredient. More specifically, the present invention relates to a pharmaceutical composition exhibiting an excellent anticancer effect and an cancer metastasis suppression effect by simultaneously targeting a cancer cell and a tumor associated macrophage (TAM) in which IL-4 receptors are over-expressed. The pharmaceutical composition of the present invention simultaneously targets a tumor cell and a TAM and kills the cells, thereby simultaneously having an anticancer effect and a cancer metastasis suppression effect. In addition, the fusion peptide of the present invention decreases side effects of conventional anticancer drugs and exhibits anticancer and cancer metastasis suppression effects, when co-administered with conventional anticancer drugs.

### CROSS REFERENCE TO RELATED PATENT APPLICATIONS

This application is a continuation of and claims priority to PCT/KR2015/012162, filed Nov. 12, 2015, which is hereby incorporated herein by reference in its entirety and which claims priority from Korean Patent Application No. 10-2015-0093576, filed on Jun. 30, 2015, which is hereby incorporated herein by reference in its entirety.

### TECHNICAL FIELD

The present invention relates to a pharmaceutical composition for treating cancer and suppressing cancer metastasis, comprising, as an active ingredient, a fusion peptide which simultaneously targets cancer cells and tumor-associated macrophages. In particular, the present invention relates to a method for treating cancer and suppressing cancer metastasis by using a fusion peptide of a IL-4 receptor-targeted peptide and a pro-apoptotic peptide, and a use of such a fusion peptide for preparing an agent for treating cancer and suppressing cancer metastasis. More particularly, the present invention relates to a pharmaceutical composition which simultaneously targets IL-4 receptor overexpressing cancer cells and tumor-associated macrophages (TAM), thereby exhibiting an excellent anticancer effect and cancer metastasis-suppressing effect.

### BACKGROUND ART

The microenvironment around the tumor is composed of endothelial cells, inflammatory cells and fibroblasts. In the 1970s, it was found that tumor-associated macrophages (TAMs) play an important role in tumor growth. TAM plays an important role in the overall tumor microenvironment such as cancer growth and metastasis, while TAM present around the tumor is closely related to tumor cell growth and metastasis. Therefore, it has been reported that the prognosis and survival rate of patients are poor if a large number of TAMs are present around the tumor in cancer patients. Among macrophages, TAM is categorized as M2 type macrophages. Unlike the common inflammatory macrophage, i.e. M1 type macrophage, M2 type macrophage produces cytokines, such as IL-10, TGF β, and CCL18, which promote the growth of cancer. In addition, receptors such as PDL1 and B7-1/2 present on the surface of M2 type TAM have been reported to inhibit the antitumor activity of T cells and NK cells. Therefore, tumor growth, differentiation and metastasis actively occur in a microenvironment where a large amount of M2 type TAM exist.

Interleukin-4 (IL-4) is a cytokine with a variety of immunoregulatory functions which is secreted by T-helper2 (Th2) lymphocytes, eosinophils, mast cells and the like. IL-4 receptor is present on the surface of such cells as T lymphocytes, B lymphocytes, and CD34 bone marrow cells among normal cells (Nelms, Annu Rev Immunol, 1999; 17: 701-738). There exist two forms of IL-4 receptor composite, i.e. Type 1 IL-4 receptor composite of the IL-4 receptor a chain and IL-2 receptor γ c chain, and Type 2 IL-4 receptor composite of IL-4 receptor a chain and IL-13 receptor al chain. The binding of IL-4 to its receptor phosphorylates and activates STAT6 signaling protein through intracellular janus kinase, while the activated STAT6 translocates to the nucleus in the form of a duplex and then regulates the expression of several IL-4 associated genes, resulting in increasing inflammation. In addition, AKT/PKB activation through janus kinase increases cell survival (Nelms et al., Annu Rev Immunol, 1999; 17: 701-738). IL-4 induces the differentiation of naive T-helper (naive Th) into Th2 lymphocytes and promotes the production of such cytokines as IL-4, IL-5, IL-9 and IL-13. It also induces the secretion of IgE (immunoglobin E) by B lymphocytes.

it has been recently reported that IL-4 is also synthesized in tumor cells and cancer stem cells, and confers tumor cell resistance to apoptosis through IL-4 receptor on the surface of cancer cells (Todaro, CellDeath Differ, 2008; 15: 762-772 Todaro, Cell Stem Cell, 2007, 1: 389-402). IL-4 receptor is greatly over-expressed in various cancer cells such as non-small cell lung cancer, brain tumor, breast cancer, bladder cancer, pancreatic cancer, kidney cancer, prostate cancer, kidney cancer and caposi's sarcoma, in comparison with normal cells. The IL-4 receptor is thus a promising target for tumor markers, in view of cancer cell resistance to anticancer agents by the IL-4 receptor and its high expression level in cancer cells.

As described above, since M2 type TAM plays an important role in tumor growth, differentiation, and metastasis, it is more desirable to develop a therapeutic agent which targets both tumor cells and M2 type TAM, instead of an agent targeting either tumor cells or M2 type TAM alone.

### DETAILED DESCRIPTION OF INVENTION

### Technical Problem

Accordingly, the inventors of the present invention have been studying a composition for treating cancer which can simultaneously target cancer cells and tumor-associated macrophages, and have found that a fusion peptide of a pro-apoptotic peptide and a peptide specifically binding to IL-4 receptor, which is highly expressed in both cancer cells and tumor-associated macrophages, effectively inhibits tumor-associated macrophages as well as cancer cells, thereby exhibiting excellent anticancer effects and cancer metastasis suppressing effects so as to complete the present invention.

Accordingly, an aspect of the present invention is to provide a pharmaceutical composition for treating cancer or suppressing cancer metastasis, the composition comprising, as an active ingredient, a fusion peptide in which a peptide specifically targeting an interleukin-4 (IL-4) receptor having the amino acid sequence of SEQ ID NO: 1 is linked to a pro-apoptotic peptide.

Another aspect of the present invention is to provide a method for treating cancer or suppressing cancer metastasis, the method comprising administering to a subject in need thereof an effective amount of a fusion peptide in which a peptide specifically targeting an interleukin-4 (IL-4) receptor having the amino acid sequence of SEQ ID NO: 1 is linked to a pro-apoptotic peptide.

Still another aspect of the present invention is to provide a fusion peptide in which a peptide specifically targeting an interleukin-4 (IL-4) receptor having the amino acid sequence of SEQ ID NO: 1 is linked to a pro-apoptotic peptide, for the preparation of an agent for treating cancer or suppressing cancer metastasis.

### Technical Solution

An embodiment according to the present invention provides a pharmaceutical composition for treating cancer or suppressing cancer metastasis, the composition comprising, as an active ingredient, a fusion peptide in which a peptide specifically targeting an interleukin-4 (IL-4) receptor having the amino acid sequence of SEQ ID NO: 1 is linked to a pro-apoptotic peptide.

Another embodiment according to the present invention provides a method for treating cancer or suppressing cancer metastasis, the method comprising administering to a subject in need thereof an effective amount of a fusion peptide in which a peptide specifically targeting an interleukin-4 (IL-4) receptor having the amino acid sequence of SEQ ID NO: 1 is linked to a pro-apoptotic peptide.

Still another embodiment according to the present invention provides a fusion peptide in which a peptide specifically targeting an interleukin-4 (IL-4) receptor having the amino acid sequence of SEQ ID NO: 1 is linked to a pro-apoptotic peptide, for the preparation of an agent for treating cancer or suppressing cancer metastasis.

Hereinafter, the present invention will be described in detail.

The present invention relates to a pharmaceutical composition for treating cancer and suppressing cancer metastasis, the composition comprising, as an active ingredient, a fusion peptide in which a peptide specifically targeting an interleukin-4 (IL-4) receptor having the amino acid sequence of SEQ ID NO: 1 is linked to a pro-apoptotic peptide.

In the present invention, the peptide (IL4RPep-1) having the amino acid sequence (CRKRLDRNC) of SEQ ID NO: 1 is a peptide specifically binding to the IL-4 receptor (IL4R). According to one example of the present invention, an immunostaining was performed using anti-IL4R antibody to macrophages on the mouse-derived 4T1 cell, the human tumor cell A549 cell line, the MDA-MB231 cell line, the M1 type Raw 264.7 macrophage, the M2 type Raw 246.7 macrophage, the mouse spleen-derived M1 type macrophage and the M2 type macrophage, respectively. As a result, it was verified that IL-4 receptors were overly expressed in the MDA-MB231 cell line, the M2 type Raw 246.7 macrophage, and the mouse spleen-derived M2 type macrophage. Subsequently, upon checking whether IL4RPep-1 (SEQ ID NO: 1) of the present invention specifically binds to IL-4 receptor, it was found that the IL4RPep-1 (SEQ ID NO: 1) of the present invention bound to the above described cells in a similar tendency to the expression of IL-4 receptor in the cells, verifying that the IL4RPep-1 (SEQ ID NO: 1) specifically binds to the IL-4 receptor (See Example 1).

According to another embodiment of the present invention, the IL4RPep-1 of the present invention exhibited a stronger binding affinity to the M2 type macrophage than the M1 type macrophage, verifying that the IL4RPep-1 specifically binds to the IL-4 receptor and can be thus used as a target-oriented drug delivery system for M2 type macrophages (See Example 1).

The peptide specifically targeting an interleukin-4 receptor of the present invention may be a functional equivalent to a peptide to which an amino acid chain is linked and preferably includes a functional equivalent to a peptide having the amino acid sequence of SEQ ID NO: 1. As used herein, “the functional equivalent” means a peptide having at least 60%, preferably 70%, more preferably 80% or more, and most preferably 90% or more sequence homology or identity with the amino acid sequence of SEQ ID NO: 1 as a result of amino acid addition, substitution or deletion, while exhibiting the substantially homogeneous activity of the peptide having the amino acid sequence of SEQ ID NO: 1 of the present invention. As used herein, “the substantially homogenous activity” means an ability of the peptide to bind to the IL-4 receptor. Such functional equivalents include, for example, amino acid sequence variants in which some of the amino acids of the peptide having the amino acid sequence of SEQ ID NO: 1 are substituted, deleted or added. Substitution of amino acids is preferably a conservative substitution. Examples of conservative substitutions of amino acids present in nature include substitution of an amino acid to another amino acid within each group, among aliphatic amino acid group (Gly, Ala, Pro), hydrophobic amino acid group (Ile, Leu, Val), aromatic amino acid group (Phe, Tyr, Trp), acidic amino acid group (Asp, Glu), basic amino acid group (His, Lys, Arg, Gin, Asn) and sulfur-containing amino acid group. The deletion of the amino acid preferably means the deletion of an amino acid located at a site which is not directly involved in the activity of the peptide having the amino acid sequence of SEQ ID NO: 1. The addition of an amino acid refers to the addition of an amino acid to an extent that such an addition does not affect the activity of the peptide, including restriction enzyme sites for genetic engineering and histidine tags for purification and the like.

In the present invention, “the pro-apoptotic peptide” refers to a peptide which induces apoptosis. Almost all cells contain mechanisms involved in mediating apoptosis. Accordingly, the present invention relates to the target delivery of a specific apoptosis-inducing peptide which is a central mediator that mediates such an effect inside the target cell and thereby kills the cell through apoptosis mechanism. As an advantage over the methods known in the art, the pro-apoptotic peptide of the present invention is delivered as a protein, not as a nucleic acid molecule which is to be decoded to produce a desired polypeptide. As further advantages, human sequences may be used in the fusion peptides of the present invention to avoid any undesired immune response by foreign polypeptides, and the pro-apoptotic peptide of the present invention can be delivered to cancerous cells in a targeted fashion. Therefore, there is an advantage that undesired adverse side effects can be alleviated.

The pro-apoptotic peptide of the present invention may be selected from the group consisting of SEQ ID NO: 2 (KLAKLAKKLAKLAK), SEQ ID NO: 4 (KGGGQVGRQLAIIGDDINR; Bak BH3 peptide), SEQ ID NO: 5 (LQHRAEVQIARKLQCIADQFHRLHT; Bmf BH3 peptide) and SEQ ID NO: 6 (YGRELRRMSDEFVDS; Bad BH3 peptide), but are not limited thereto. Those skilled in the art will be familiar with pro-apoptotic peptides, including those not specifically mentioned herein.

Preferably, the pro-apoptotic peptide of the present invention may be a peptide having the amino acid sequence of SEQ ID NO: 2 (KLAKLAKKLAKLAK).

In the present invention, the pro-apoptotic peptide having the amino acid sequence of SEQ ID NO: 2 may be composed of L-type or D-type amino acids in consideration of its stability in the body.

The present invention provides a fusion peptide in which the peptide having the amino acid sequence of SEQ ID NO: 1 is linked to the pro-apoptotic peptide, and the peptides of the present invention can be produced by a method known to a person skilled in the art. Such peptides can be produced in prokaryotic or eukaryotic cells by expressing polynucleotides encoding the peptide sequences of the invention, often as a part of larger polypeptides.

Alternatively, such peptides can be synthesized by chemical methods. Methods for the expression of heterologous proteins in recombinant hosts, the chemical synthesis of polypeptides and in vitro transcription are well known in the art and are described in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Ann. Rev. Biochem. 57:957; and Offord, R. E. (1980) Semisynthetic Proteins, Wiley Publishing.

In the present invention, the fusion peptide may be a fusion peptide in which the peptide having the amino acid sequence of SEQ ID NO: 1 is linked to the pro-apoptotic peptide through a linker. The linker may be present between the C-terminus of the peptide having the amino acid sequence of SEQ ID NO: 1 and the N-terminus of the pro-apoptotic peptide.

The linker is inserted in the process of preparing a polynucleotide encoding the fusion polypeptide of the present invention, and its size and sequence type are not particularly limited.

The linker may increase the activity of the fusion peptide by minimizing the potential interference of the two peptides. The linker preferably has 1 to 100 amino acids, but is not limited thereto, and any peptide capable of linking and separating two peptides may be used. The amino acid sequence constituting the linker is not particularly limited, but may preferably be a peptide linker composed of at least one amino acid selected from the group consisting of alanine, glycine, and combinations thereof. That is, it may be a linker composed of alanine, a linker composed of glycine or a linker composed of alanine and glycine. For such amino acids, those may be selected which contain no functional group and thus non-specific binding does not occur, as well as no problem in folding.

In addition, the linker according to the present invention does not interfere with either the activity of the peptide having the amino acid sequence of SEQ ID NO: 1 binding to the IL-4 receptor, or the activity of the pro-apoptotic peptide, while being composed of an appropriate number of amino acids providing a flexibility for maintaining a proper orientation.

In one example of the present invention, the linker as used is a linker in which three glycines are successively combined.

In one example of the present invention, a fusion peptide was prepared in which the peptide having an amino acid sequence of SEQ ID NO: 1, which binds to the IL-4 receptor, the linker and the pro-apoptotic peptide are linked sequentially, followed by the analysis of its activity. The fusion peptide has the amino acid sequence of SEQ ID NO: 3 (CRKRLDRNCGGGKLAKLAKKLAKLAK).

The pharmaceutical composition according to the present invention may be formulated into a suitable form which comprises the fusion peptide alone or in combination with a pharmaceutically acceptable carrier, and may further contain an excipient or a diluent. The term “pharmaceutically acceptable” as used herein refers to a non-toxic composition that is physiologically acceptable and does not normally cause an allergic reaction such as gastrointestinal disorder and dizziness or similar reactions when administered to humans.

The pharmaceutically acceptable carrier may further include, for example, a carrier for oral administration or a carrier for parenteral administration. Carriers for oral administration may include lactose, starch, cellulose derivatives, magnesium stearate, stearic acid, and the like. In addition, it may contain various drug delivery materials used for oral administration of peptide preparations. In addition, the carriers for parenteral administration may contain water, a suitable oil, saline solution, an aqueous glucose, glycol and the like, and may further contain a stabilizer and a preservative. Suitable stabilizers are antioxidants such as sodium hydrogen sulfite, sodium sulfite or ascorbic acid. Suitable preservatives include benzalkonium chloride, methyl- or propyl-paraben and chlorobutanol. The pharmaceutical composition of the present invention may further contain a lubricant, a wetting agent, a sweetener, a flavoring agent, an emulsifying agent, a suspending agent, etc., in addition to the above components. Other pharmaceutically acceptable carriers and preparations can be found in Remington's Pharmaceutical Sciences, 19th ed., Mack Publishing Company, Easton, Pa., 1995.

The composition of the present invention can be administered to mammals including humans by any method. For example, it can be administered orally or parenterally. Parenteral administration methods include, but are not limited to, intravenous, intramuscular, intraarterial, intramedullary, intrathecal, intracardiac, transdermal, subcutaneous, intraperitoneal, intranasal, enteral, topical, sublingual or rectal administration.

The pharmaceutical composition of the present invention may be formulated into oral or parenteral dosage forms according to the route of administration as described above.

In the case of oral preparations, the composition of the present invention may be formulated into powder, granules, tablets, pills, sugar-coated tablets, capsules, liquids, gels, syrups, slurries, suspensions or the like by methods known in the art. For example, an oral preparation can be obtained by combining the active ingredient with a solid excipient, then milling it, adding suitable excipients, and then processing the mixture into a granular mixture to obtain tablets or sugar-coated tablets. Examples of suitable excipients include sugars such as lactose, dextrose, sucrose, sorbitol, mannitol, xylitol, erythritol and maltitol, and starches such as corn starch, wheat starch, rice starch and potato starch, cellulose such as methylcellulose, sodium carboxymethylcellulose and hydroxypropylmethyl-cellulose, fillers such as gelatin and polyvinylpyrrolidone. In addition, crosslinked polyvinylpyrrolidone, agar, alginic acid or sodium alginate may optionally be added as a disintegrant. Furthermore, the pharmaceutical composition of the present invention may further comprise an anti-coagulant, a lubricant, a wetting agent, a flavoring agent, an emulsifying agent and an antiseptic agent.

The preparation for parenteral administration may be formulated into injections, creams, lotions, topical ointments, oils, moisturizers, gels, aerosols and nasal inhalers by methods known in the art. These formulations are described in Remington's Pharmaceutical Science, 19th ed., Mack Publishing Company, Easton, Pa., 1995, which is a commonly known formulary for all pharmaceutical chemistries.

The total effective amount of a composition of the present invention may be administered to a patient in a single dose and may be administered by a fractionated treatment protocol administered over a prolonged period of time in multiple doses. The pharmaceutical composition of the present invention may contain a varied amount of the active ingredient depending on the severity of disease. Particularly, the preferred total amount of the pharmaceutical composition of the present invention may be from about 0.01 μg to about 10,000 mg per kg body weight per day, and most preferably from 0.1 mg to 500 mg per kg body weight per day. However, the dosage of the pharmaceutical composition may be determined depending on various factors such as the formulation method, administration route and frequency of treatment, as well as the patient's age, weight, health condition, sex, severity of disease, diet and excretion rate. One of ordinary skill in the art will be able to determine the appropriate effective dose of the composition of the present invention in view of those factors. The pharmaceutical composition according to the present invention is not particularly limited to specific formulations, administration routes and administration methods as long as the effect of the present invention is exhibited.

The composition comprising the fusion peptide of the present invention as an active ingredient exhibits excellent anticancer and cancer metastasis inhibitory effects. Specifically, in one example of the present invention, a fusion peptide (IL4RPep-1-KLA peptide; SEQ ID NO: 3) in which the peptide having the amino acid sequence of SEQ ID NO: 1 (IL4RPep-1) is linked to the pro-apoptotic peptide (KLA; SEQ ID NO: 2) were administered to mouse 4T1 tumor cells and mouse tumor model transplanted with such tumor cells, respectively. As a result, it was found that the fusion peptide exhibited excellent cancer cell death and growth inhibition effects in vitro and in vivo, confirming its remarkable anticancer effect (See Example 5).

Furthermore, in in vivo mouse tumor model, following the administration of the IL4RPep-1-KLA peptides (SEQ ID NO: 3), the lungs and liver of the animal model were excised for observing the existence of tumor metastasis. While PBS-administered control group showed a significant tumor metastasis in the lungs and liver, no tumor metastasis was observed in the mouse group to which the IL4RPep-1-KLA peptide (SEQ ID NO: 3) was administered, verifying that the fusion peptide of the present invention is also excellent in suppressing tumor metastasis (See Example 5).

The fusion peptides of the present invention exhibits excellent anti-cancer and cancer metastasis inhibitory effects since they are capable of targeting the IL-4 receptors overexpressed on the surface of tumor cells and tumor-associated macrophages and delivering the pro-apoptotic peptides thereto. In other words, unlike existing anticancer therapeutic agents targeting only tumor cells to induce their death, the fusion peptides of the present invention are capable of further targeting and killing the tumor-associated macrophages which play a very important role in tumor growth, differentiation and metastasis (See Example 5). Thus, the fusion peptides of the present invention are excellent in cancer metastasis inhibition as well as anti-cancer.

In this way, the pharmaceutical composition according to the present invention that simultaneously targets tumor cells and tumor-associated macrophages and exhibits excellent anti-cancer and cancer metastasis inhibitory effects is considered to be a novel targeted therapeutic agent that has not been reported so far.

In another example of the invention, it was verified that the peptide (IL4RPep-1) having the amino acid sequence of SEQ ID NO: 1, which plays a role as a targeted delivery system of a therapeutic agent in the fusion polypeptide of the invention, showed high specificity and binding affinity to IL-4 receptors in vitro and in vivo (See Examples 1 and 2).

Accordingly, the present invention provides a pharmaceutical composition characterized in that the cancer is cancer in which the IL-4 receptors are overly expressed.

More specifically, the cancer over-expressing the IL-4 receptors is selected from the group consisting of lung cancer, brain tumor, breast cancer, liver cancer, skin cancer, esophageal cancer, testicular cancer, kidney cancer, colon cancer, rectal cancer, stomach cancer, bladder cancer, ovarian cancer, cholangiocarcinoma, gallbladder cancer, uterine cancer, cervical cancer, prostate cancer, head and neck cancer, pancreatic cancer, and squamous cell carcinoma, and is not limited thereto.

The present invention also provides a pharmaceutical composition, wherein said composition is co-administered with an anti-cancer drug.

More specifically, the anticancer drug may be selected from the group consisting of doxorubicin, paclitaxel, vincristine, daunorubicin, vinblastine, actinomycin-D, docetaxel, etoposide, teniposide, bisantrene, homoharringtonine, Imatinib (Gleevec; STI-571), cisplatin, 5-fluorouracil, adriamycin, methotrexate, busulfan, chlorambucil, cyclophosphamide, melphalan, nitrogen mustard, and nitrosourea, and is not limited thereto.

In one example of the present invention, paclitaxel, which is widely used as an anticancer agent, was treated in a dose which does not show a sufficient therapeutic effect by its single administration, together with the IL4RPep-1-KLA fusion peptide (SEQ ID NO: 3). As a result, it was found that the IL4RPep-1-KLA fusion peptide (SEQ ID NO: 3) can be administered together with the existing anticancer drug as a combination drug that can maximize such a therapeutic effect (See Example 5).

As used herein, the term “co-administration” or “administration in combination” means that two or more agents can be found in the bloodstream of a patient at a specific time, regardless of when and how they are actually administered. The co-administration can be carried out by administering the fusion peptide of the present invention and the anti-cancer drug together or sequentially. The co-administration can be also carried out by administering a pharmaceutical composition in which an effective amount of the fusion peptide of the present invention and the anti-cancer drug are mixed. In another embodiment, the co-administration may be conducted by performing simultaneously or sequentially a first step of administering a pharmaceutically effective amount of the fusion peptide of the invention and a second step of administering a pharmaceutically effective amount of the anti-cancer drug. When a sequential administration is performed, the order of administration may be reversed. In other embodiments, the fusion peptide and the anti-cancer drug may be administered via the same route such as orally, intravenously, etc., or via different routes, such as oral administration of one active ingredient and intravenous administration of the other active ingredient.

The present invention relates to a method for treating cancer or suppressing cancer metastasis, the method comprising administering to a subject in need thereof an effective amount of the fusion peptide in which the peptide specifically targeting an interleukin-4 (IL-4) receptor having the amino acid sequence of SEQ ID NO: 1 is linked to the pro-apoptotic peptide.

The present invention relates to the fusion peptide, in which the peptide specifically targeting an interleukin-4 (IL-4) receptor and having an amino acid sequence of SEQ ID NO: 1 is linked to the pro-apoptotic peptide, for use in the preparation of an agent for treating cancer or suppressing cancer metastasis.

The term “an effective amount” as used herein means, when administered to a patient, an amount which induces the effects of treating or preventing cancer or suppressing cancer metastasis. The term “subject” refers to an animal, preferably a mammal including a human in particular, while including animal-derived cells, tissues, organs, and the like. The subject may be a patient requiring such a treatment.

### Advantageous Effects

As described above, the pharmaceutical composition comprising the fusion peptide of the present invention as an active ingredient has an effect of simultaneously targeting and killing tumor cells and tumor-associated macrophages, and has thus excellent anti-cancer and cancer metastasis inhibitory effects, while its combination therapy with existing anti-cancer agents reduces the side effects of conventional anti-cancer agents and also has anti-cancer and cancer metastasis inhibitory effects.

### BRIEF DESCRIPTION OF DRAWINGS

FIG. 1 shows immunofluorescence results in which tumor cells (4T1, A549, MDA MB231) and Raw 264.7 macrophages (M1 type, M2 type) were immunostained with anti-IL4R α, anti-IL13Rα, and anti-IL2γC antibodies, respectively, and the expression level of each receptors were observed, while the binding degrees of the IL4RPep-1 peptide (SEQ ID NO: 1) to each cells were detected. Results with a control peptide (NSSSVDK; SEQ ID NO: 7) are also shown.

FIG. 2A shows immunofluorescence results in which the mouse spleen-derived macrophages were differentiated into M1 type and M2 type macrophages, respectively and then the expression level of IL-4 receptors and the binding degree of the IL4RPep-1 peptide (SEQ ID NO: 1) to each cell were observed.

FIG. 2B shows results of the binding affinity of the IL4RPep-1 peptide (SEQ ID NO: 1) to M1 type and M2 type macrophages, respectively, as calculated by Graph Pad Prism 6 software.

FIGS. 3A and 3B show fluorescent imaging results in which Flamma 675-labeled control peptide (SEQ ID NO: 7 (NSSSVDK)) or IL4RPep-1 peptide (SEQ ID NO: 1) were intravenously administered to 4T1 tumor cells-transplanted wild-type Balb/c mice (Balb/c WT mice) and IL-4 receptor deficient Balb/c mice (Balb/c IL4R α K/O mice), respectively, followed by the detection of fluorescent imaging in said mice (in vivo) 1 to 2 hours after said administration or in the excised organs of said mice after the completion of the experiment (ex vivo) [FIG. 3A: control peptide (SEQ ID NO: 7)-treated mice group, FIG. 3B: IL4RPep-1 peptide (SEQ ID NO: 1)-treated mice group].

FIGS. 4A and 4B show immunofluorescent imaging results in which the IL4RPep-1 peptides (SEQ ID NO: 1) was intravenously administered to 4T1 tumor cells-transplanted wild-type Balb/c mice (Balb/c WT mice) and IL-4 receptor deficient Balb/c mice (Balb/c IL4R α K/O mice), respectively, followed by the excision and fragmentation of tumor tissues of said mice for observing the expressions of IL-4 receptor, F4/80 (tumor-associated macrophage marker), E-cadherin (epithelial cell marker) and N-cadherin (mesenchymal marker) [FIG. 4A: tumor tissues from wild-type Balb/c mice (Balb/c WT mice), FIG. 4B: IL-4 receptor deficient Balb/c mice (Balb/c IL4R α K/O mice)].

FIG. 5 shows immunofluorescent staining results of observing the expression of IL-4 receptor, E-cadherin (epithelial cell marker) and N-cadherin (mesenchymal marker) of 4T1 tumor cells, respectively.

FIG. 6 shows flow cytometry (FACS) analysis results of N-cadherin, F4/80 and E-cadherin expressed on the surface of a single cell suspension of the tumor excised from 4T1 tumor cells-tansimplanted mice [Ncad: N-cadherin, Ecad: E-cadherin].

FIG. 7 shows immunofluorescent staining results of observing the expressions of N-cadherin and IL-4 receptor after treating 4T1 tumor cells with tumor-associated macrophages (TAM)-conditioned media, TGF β, IL10 and IL4, respectively [TAM CM: tumor-associated macrophages-conditioned media, Ncad: N—cadherin].

FIG. 8 shows results of measuring the IL-10 secretion by using IL-10 ELISA kit after mouse spleen-derived M1 type or M2 type macrophages were treated according to each condition [CM: conditioned media].

FIG. 9A shows results of measuring the binding affinity of the IL4RPep1 peptide (SEQ ID NO: 1) to wild-type 4T1 cells and IL-10-treated 4T1 cells, respectively.

FIG. 9B shows immunofluorescent staining results of the expression levels of IL-4 receptor, IL-13 receptor and IL-2 receptor, respectively, and the binding degrees of the control peptide (NSSSVDK; SEQ ID NO: 7) and the IL4RPep-1 peptide (SEQ ID NO: 1) in the 4T1 cells treated by M2 type macrophage exosomes, respectively.

FIGS. 10A-10D show cytotoxicity results of the IL4RPep-1-KLA fusion peptides (SEQ ID NO: 3; IL4Rpep1KLA) [FIG. 10A: cytotoxicity results for the wild-type 4T1 cells, FIG. 10B: cytotoxicity results for the IL-10-treated 4T1 cell, FIG. 10C: cytotoxicity results for M1 type macrophages, FIG. 10D: cytotoxicity results for M2 type macrophage].

FIG. 11 shows experimental results of evaluating the anti-cancer effect of IL4RPep-1-KLA (SEQ ID NO: 3) and its co-administration effect with paclitaxel in the 4T1 tumor cells-transplanted mouse animal models [IL4RPep-1, KLA: a group in which the IL4RPep-1 peptide (SEQ ID NO: 1) and the KLA peptide (SEQ ID NO: 2) were individually administered, IL4RPep-KLA: the fusion polypeptide (SEQ ID NO: 3)-administered group, PTX: paclitaxel-administered group, IL4RPep-1, KLA+PTX: a group in which IL4RPep-1 peptide (SEQ ID NO: 1), and KLA peptide (SEQ ID NO: 2) and paclitaxel were individually administered, IL4R-Pep-KLA+PTX: a group in which the fusion polypeptide (SEQ ID NO: 3) and paclitaxel were co-administered].

FIGS. 12A and 12B show results of counting post-administration metastatic tumor nodules in mouse lungs and liver of each drug administration group in the 4T1 tumor cells-transplanted mouse animal models [IL4RPep-1, KLA: a group in which the IL4RPep-1 peptide (SEQ ID NO: 1) and the KLA peptide (SEQ ID NO: 2) were individually administered, IL4RPep-KLA: the fusion polypeptide (SEQ ID NO: 3)-administered group, PTX: paclitaxel-administered group, IL4RPep-1, KLA+PTX: a group in which IL4RPep-1 peptide (SEQ ID NO: 1), and KLA peptide (SEQ ID NO: 2) and paclitaxel were individually administered, IL4R-Pep-KLA+PTX: a group in which the fusion polypeptide (SEQ ID NO: 3) and paclitaxel were co-administered].

FIG. 13 shows microscopic results in which frozen sections of tumor tissues of each group after administration in the 4T1 tumor cells-transplanted mouse animal models were prepared and subsequently stained with corresponding antibodies [E-cadherin, N-cadherin, F4/80, CD80, CD8 T cell and CD4 T cell) (IL4RPep-1, KLA: a group in which the IL4RPep-1 peptide (SEQ ID NO: 1) and the KLA peptide (SEQ ID NO: 2) were individually administered, IL4RPep-KLA: the fusion polypeptide (SEQ ID NO: 3)-administered group, PTX: paclitaxel-administered group, IL4RPep-1, KLA+PTX: a group in which the IL4RPep-1 peptide (SEQ ID NO: 1), and the KLA peptide (SEQ ID NO: 2) and paclitaxel were individually administered, IL4R-Pep-KLA+PTX: a group in which the fusion polypeptide (SEQ ID NO: 3) and paclitaxel were co-administered).

### MODE FOR CARRYING OUT INVENTION

Hereinafter, the present invention will be described in detail.

The following examples are merely illustrative of the present invention, and the present invention is not limited to the following examples of this disclosure.

<Experimental Methods>

1. Cell Lines and Culture

Mouse tumor cells 4T1, mouse macrophages Raw 264.7, human tumor cells A549 and MDA MB 231 cell line were cultured in Dulbecco's modified Eagle's medium (Gibco, USA) or RPMI medium in accordance with the instructions of the ATCC.

Spleen-derived macrophages were extracted according to Alatery et al.'s methods (See Journal of immunological methods, 2008. 338 (1): p. 47-57)

2. The Differentiation of Macrophages into M1 Type and M2 Type Ones

The M2 type macrophages, which are tumor-associated macrophages, were able to be obtained by treating Raw 264.7 cells and/or mouse spleen-derived macrophages with 10 IU/ml of mouse recombinant IL-4 (R and D system, US), while the M1 type macrophages by treating with 100 IU/ml of IFN-γ (R and D system, US) and 10 ng/ml of LPS (Sigma-aldrich).

For the purpose of verifying the successful completion of such a differentiation, anti-F4/80 and/or anti-CD 163 antibodies were used for M2 type macrophages, while anti-CD80 antibodies were used for M1 type macrophages.

3. Production of the Fusion Peptide

For in vitro experiment, fluorescein isothiocyanate (FITC) or biotin was bound to the N-terminus of the IL4RPep-1 peptide having the amino acid sequence (CRKRLDRNC) of SEQ ID NO: 1 that specifically binds to IL-4 receptor. The IL4RPep-1 peptide (SEQ ID NO: 1) bound to Flamma 675 was used in in vivo optical imaging experiment. A peptide having an amino acid sequence (NSSSVDK) of SEQ ID NO: 7 was used as a control peptide.

The IL4RPep-1-KLA fusion protein (SEQ ID NO: 3) was prepared by fusing the IL4RPep-peptide 1 peptide of SEQ ID NO: 1 with the pro-apoptotic peptide of KLAKLAKKLAKLAK (SEQ ID NO: 2, hereinafter “KLA”) via a triple glycine linker.

All peptides were synthesized by Peptron Inc. (Daejon, Republic of Korea) with a purity of 90% or greater as purified by high purity liquid chromatography (HPLC). The peptides were freeze-dried and dissolved in PBS before their use.

4. Immunofluorescent Staining of IL4RPep-1 Bound to IL-4 Receptors and Cells

In order to test whether the peptides binds to cells, tumor cells were blocked with 1% BSA solution initially, 10 μM FITC-labeled IL4RPep1 peptides of SEQ ID NO: 1 were then incubated at 4° C. for 1 hour. The cells were washed and fixed with 4% PFA, while their nuclei were stained with DAPI.

To evaluate the expression level of IL-4 receptor in the cells or tumor tissues, immunostaining was performed on frozen tissue sections or fixed mouse tumor cell lines by using anti-IL-4R, anti-IL13Rα1 and anti-IL2R γC antibodies.

After being washed with PBS, the cells were incubated at room temperature for 1 hour with secondary antibodies. Finally, following nuclear staining with DAPI, the cells were examined by a fluorescence microscope (Zeiss, Germany).

5. IL4RPep-1 Binding Affinity Assay

Blocking the tumor cells with 1% BSA at room temperature for 30 minutes, the biotin-labeled IL4RPep-1 peptides of various concentrations (1 to 80 μM) were incubated for 1 hour. After washing with PBS, the cells were incubated with Neutravidin HRP (1: 10000) at room temperature for 30 minutes. HRP activity was measured by using the TMB substrate, and the reaction was stopped using 2M sulfuric acid. Absorbance was measured at 450 nm using TECAN microplate reader. Kd values were calculated using Graph Pad Prism 6 software (GraphPad software Inc., La Jolla, La.).

6. In Vivo Optical Imaging and Immunohistochemical Analysis

6-1. Animal Model

Female wild-type Balb/c mice were purchased from Orient Bio (Republic of Korea) to prepare IL-4 receptor-deficient mice. Mouse tumor models were produced by subcutaneously injecting 1×10⁶ 4T1 tumor cells into the flank top skin of the wild-type and IL-4 receptor-deficient mice, respectively. Orthotopic animal models were produced by injecting 1×10⁶ 4T1 cells into the mammary fat tissue of the mice.

6-2. In Vivo Imaging and Histological Analysis

The Flamma 675-labeled IL4RPep-1 peptide (SEQ ID NO: 1) and control peptide (NSSSVDK) of SEQ ID NO: 7 were administered via the tail vein of 4T1 tumor cells-transplanted wild-type mice and the IL-4 receptor-deficient mice, respectively. In vivo imaging was conducted using the Optix imaging system (ART Inc., Canada) after one hour and two hours circulation. The mice were sacrificed after imaging, and tumors and organs were then excised for ex vivo imaging.

For immunohistochemical analysis, excised tumors were fixed with 4% PFA and dehydrated with 30% sucrose. 8 μm thick slices of the tumor were prepared and their histological structures were evaluated by DAPI staining.

Additionally, in order to analyze the tendency of lateralization of the peptide and the receptor in the tumor tissues, the tumor samples were immunostained with anti-IL4Rα antibodies and F4/80 antibodies, followed by detection with Alexa-488/594-conjugated secondary IgG antibodies (invitrogen).

E-cadherin and N-cadherin were used as markers of the 4T1 tumor cells. Tumor sections were immunostained with ant-E-cadherin and anti-N-cadherin antibodies, followed by staining with secondary antibodies and DAPI. The cells were observed using a confocal microscope (Zeiss, Germany).

7. IL-4 Receptor Expression Analysis Using Flow Cytometry (FACS) in Single Cell Suspension of Mouse Tumor Tissues

The excised mouse 4T1 tumors were crushed mechanically using surgical scissors, and isolated by Liberase™. After isolating enzymatically, the samples were transferred to ice and the reaction was stopped. The tumor cells were stained with cell staining solution, followed by washing with FACS buffer. Subsequently, RBCs were removed by using RBC lysis buffer (Sigma), and the obtained cell precipitates were stained with the primary antibodies (anti-IL4R α, anti-F4/80, anti-E-cadherin, and anti-N-cadherin antibodies), followed by being attached to the secondary antibodies. The stained cells were analyzed using BD FACS Calibur.

8. Induced Epithelial Mesenchymal Transition of 4T1 Cancer Cells

4T1 cells were cultured using DMEM medium containing 50% of the mouse spleen-derived macrophages which were differentiated into M2 type macrophages. As for the culture medium containing TAM, the debris was removed by centrifugation and strainer prior to use. Cytokine-induced epithelial mesenchymal transition was implemented by culturing the cells with mouse IL-10 and mouse IL-4 for 24 hours or with mouse TFGβ-containing medium for 48 hours. Subsequently, the cells were stained with N-cadherin (a mesenchymal marker), and the expression level of IL-4 receptor was evaluated.

9. IL-10 Secretion Assay

IL-10 cytokines or exosomes secreted by M2 type macrophages were evaluated by mouse IL-10 ELISA kit according to the manufacturer's instructions. Absorbance was measured at 450 nm, and the concentration of IL-10 was obtained from the absorbance value using the standard curve.

10. Exosome-Induced Epithelial Mesenchymal Transition of 4T1 Cells and Evaluation of the Expression of IL-4 Receptor

Exosomes were isolated from conditioned media by using Exoquick TC kit (SBI Bioscience). 4T1 cancer cells, which were cultured in exosome-deficient DMEM culture medium containing FBS, were cultured with 50/ml of the isolated exosomes for 24 hours. Thereafter, the cells were stained for observing IL-4 receptor and EMT (epithelial-mesenchymal transition) marker N-cadherin, and were observed through a fluorescent microscope.

11. Cytotoxicity Evaluation of IL4RPep-1-KLA (SEQ ID NO: 3)

Cytotoxicity of the IL4RPep-1-KLA (SEQ ID NO: 3) was evaluated according to the manufacturer's instructions using CCK8 kit (Dojindo laboratories, Japan). IL-4 receptor-expressing A549 cells were briefly cultured with various concentration (0-160 μM) of IL4RPep-1-KLA (SEQ ID NO: 3) for 1 hour, followed by the addition of CCK solution for further culturing for 1 to 4 hours. Absorbance was measured at 450 nm, and cytotoxicity was calculated by the following equation:

Cell viability=(A sample−A blank/A control−A blank)×100

A blank=Absorbance value of the well which contains test material but does not contain cells

A control=Absorbance value of the well which contains only cells and CCK8 solution

12. In Vivo Anti-Cancer Activity Evaluation

Orthotropic 4T1 tumor model were produced by the implantation of 1×10⁶ 4T1 cells into the left mammary fat pad of the wild-type Balb/c mouse. Tumor was left to grow until its size reached approximately 100 mm³, followed by random segregation of mice into separate groups for administration. Mice were separated into a total of six groups with five rats in each group. Peptides (KLA (SEQ ID NO: 2)+IL4RPep-1 (SEQ ID NO: 1) and IL4RPep-1-KLA fusion polypeptide (SEQ ID NO: 3), respectively) were administered via the tail vein of the mouse at an equimolar concentration (1 mM peptide 200/20 g body weight of mice, administered 3 times a week for 4 weeks). Mice of the other three groups were intraperitoneally administered with 8 mg/kg dosage concentration of paclitaxel once a week, in addition to the administration of the peptides. Two control mice groups were administered with PBS or paclitaxel, respectively.

The weight and tumor size of the mice were observed after administration. The tumor size was measured with a digital caliper, and the volume of the tumor was calculated by the following equation:

V=(L×W×H)/2 (L: the longest-length, W: Short length, H: height)

The mice were sacrificed after the last dose, and metastasis of the tumor into the lungs and liver was evaluated. The excised tumors and organs were fixed in 4% PFA and used for additional immunohistological analysis.

13. Immunohistological Staining of Tumor Tissue

Frozen tumor tissues were blocked with a blocking solution of 1 g of BSA, 0.2 g of gelatin and 0.05 g of saponin dissolved in PBS, followed by incubation using primary antibodies for 1½ hours and staining using HRP-labeled secondary antibodies for 45 minutes at room temperature. The stained tissues were exposed using DAB (DAKO) and a contrast dyeing was performed using hematoxylin for 5 minutes at room temperature. Each step moved onto the next one after washing with PBS containing 10% blocking solution. Finally, the tissues were observed by Bright Field Microscopy.

### EXPERIMENTAL RESULTS (EXAMPLES)

### Example 1

In Vitro Experimental Results on the Binding of IL4RPep-1 Peptide (SEQ ID NO: 1) to IL-4 Receptor

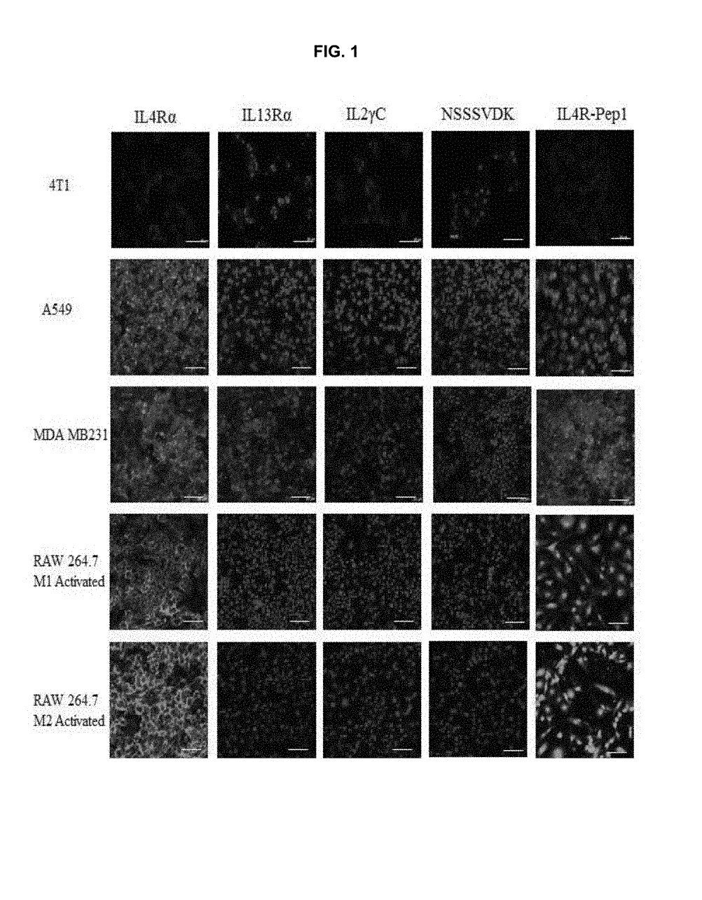

The expression levels of IL-4 receptors, IL-13 receptors and IL-2 receptors were determined in mouse 4T1 cells, human tumor cells A549, MDA-MB 231, M1 type Raw 264.7 cells, M2 type Raw 264.7 cells, mouse spleen-derived M1 type macrophages and M2 type macrophages, respectively. Subsequently, the specific binding of the peptide (IL4RPep-1) according to the present invention having the amino acid sequence of SEQ ID NO: 1 to said receptors was evaluated by immunostaining. Results are shown in FIG. 1.

As shown in FIG. 1, it was found that only IL-4 receptors among IL-4 receptors, IL-13 receptors and the IL-2 receptors were strongly stained with fluorescence in mouse 4T1 cells, human tumor cells A549, MDA-MB 231, M1 type Raw 264.7 cells and M2 type Raw 264.7 cells, verifying that IL-4 receptors were overexpressed in said cells.

Subsequently, the peptides (IL4RPep-1 peptides) according to the present invention having the amino acid sequence of SEQ ID NO: 1 were treated in said cells, resulting in the binding of the IL4RPep-1 peptides in said cells in the same manner as the expression pattern of IL-4 receptors. This result confirms that the IL4RPep-1 peptides specifically bind to IL-4 receptors.

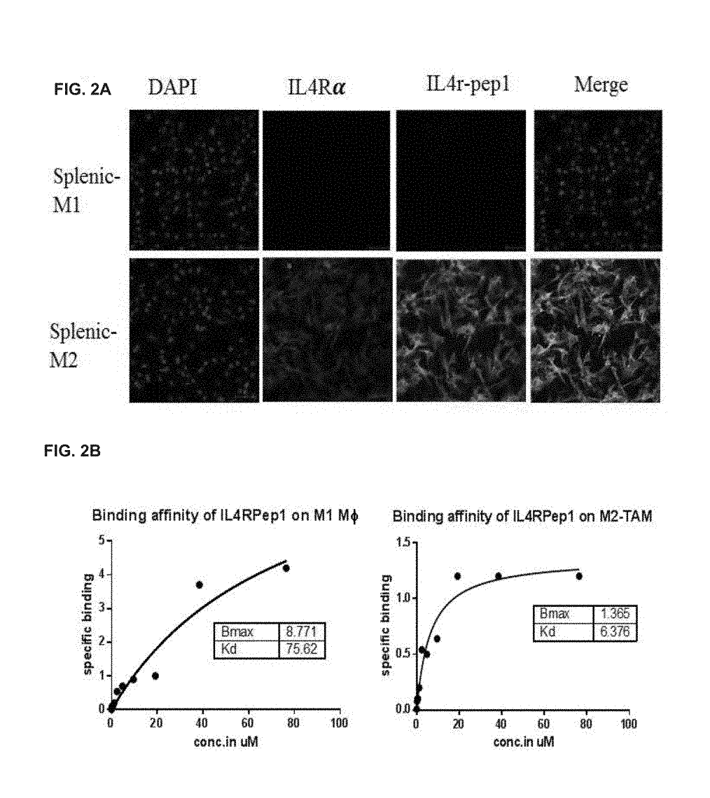

In addition, the expression pattern of IL-4 receptors and the binding pattern of the IL4RPep-1 peptides in mouse spleen-derived M1 type macrophages and M2 type macrophages were compared using immunostaining method, while the binding affinity of the IL4RPep-1 peptides to said cells was measured, respectively. Results are shown in FIGS. 2A and 2B.

As shown in FIG. 2A, it was found that IL-4 receptors were over-expressed in M2 type macrophages among the mouse spleen-derived macrophages, unlike M1 type macrophages, and that the binding pattern of the IL4RPep-1 peptides to the cells was the same as the expression pattern of IL-4 receptors. Meanwhile, as shown in FIG. 2B, it was verified that the IL4RPep-1 peptides exhibited a stronger binding affinity to M2 type macrophages, in which IL-4 receptors were over-expressed, than M1 type macrophages (M1 type macrophages: Kd 75.8; M2 type macrophages: Kd 6.3).

Through the above results, it was confirmed that the IL4RPep-1 peptide having the amino acid sequence of SEQ ID NO: 1 specifically binds to IL-4 receptor and can be thus useful for targeting IL-4 receptor as a drug delivery system, and that IL-4 receptor is over-expressed in M2 type macrophages in comparison with M1 type macrophages.

### Example 2

In Vivo Experimental Results on the Binding of IL4RPep-1 Peptide (SEQ ID NO: 1) to IL-4 Receptors

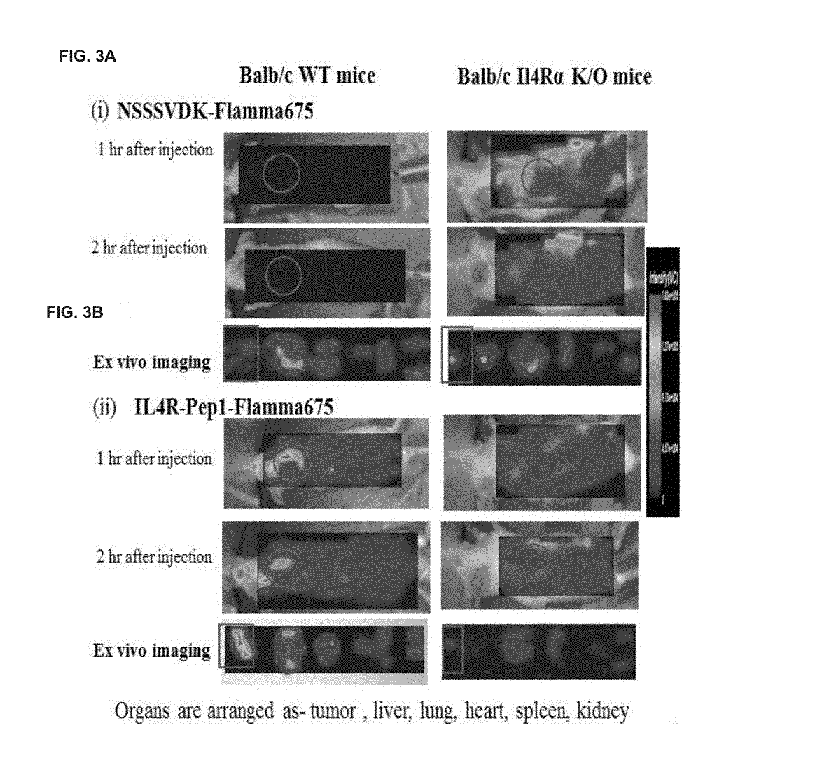

It was evaluated whether the IL4RPep-1 peptide having the amino acid sequence of SEQ ID NO: 1 specifically binds to IL-4 receptor in 4T1 tumor cells-transplanted Balb/c wild-type mice and Balb/c IL-4 receptor-deficient (knockout) mice, respectively. That is, Flamma 675-labeled IL4RPep-1 peptides and control peptides (NSSSVDK, SEQ ID NO: 7) were administered into the tail vein of the mouse, respectively, followed by real time observation for fluorescence intensity. Results are shown in FIGS. 3A and 3B.

As shown in FIGS. 3A and 3B, upon ex vivo comparing fluorescence intensity in the mouse body and excised mouse tissues, it was found that the IL4RPep-1 peptides fluorescence was strongly detected in the tumor tissues of wild-type mice which express IL-4 receptors normally, whereas no fluorescence was detected in the tumor tissues of the IL-4 receptor-deficient (knockout) mice and those of control peptide-administered mice. That is, it was confirmed in vivo that the IL4RPep-1 peptides having the amino acid sequence of SEQ ID NO: 1 specifically bind to the tumor tissues of the mice which express the IL-4 receptors.

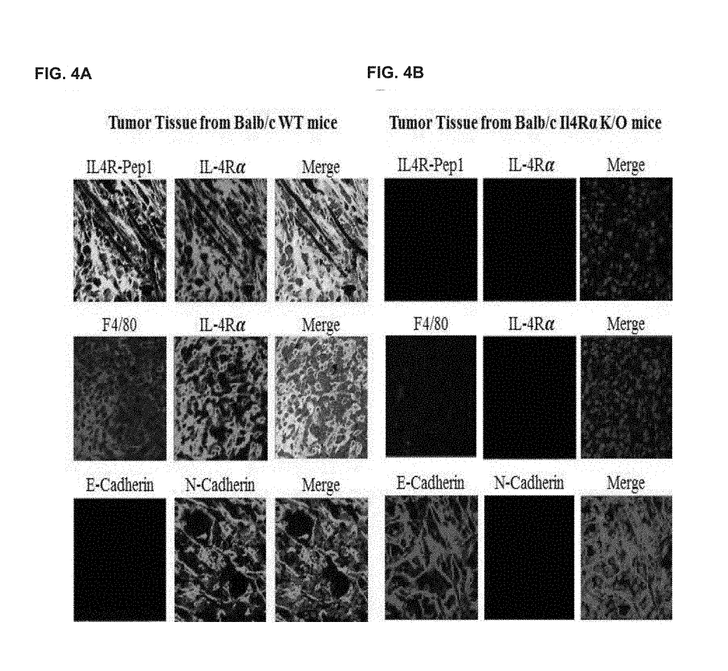

Meanwhile, immunostaining was performed on the fragments of the excised tumor tissues of the mice upon the completion of the above experiment. As shown in FIGS. 4A and 4B, it was found that IL-4 receptors were strongly stained in the tumor tissues of the wild-type mice, and that the IL4RPep-1 peptides were stained in the tumor tissues of the mice in the same pattern as that of IL-4 receptors (See FIG. 4A). On the other hand, it was shown that IL-4 receptors were not observed at all in the tumor tissues of the IL-4 receptor-deficient mice, with no binding of the IL4RPep-1 peptides (See FIG. 4B).

### Example 3

Inducement of Epithelial Mesenchymal Transition of 4T1 Tumor Cells by Tumor-Associated Macrophages (TAM) (In Vivo)

After being excised from Balb/c wild-type mice and Balb/c IL-4 receptor-deficient (knockout) mice which completed the experiments of Example 2, tumors were stained with anti-IL-4 receptor antibodies and antibodies to F4/80 known as a tumor-associated macrophage (TAM) marker. Results as observed are shown in FIGS. 4A and 4B.

As shown in FIGS. 4A and 4B, in spite of the specificity of the IL4RPep-1 peptides for TAM which expresses IL-4 receptors, it was found that there was no lateralization in comparison with the TAM marker F4/80 in a portion of the tumor tissues which were stained with anti-IL-4 receptor antibodies. That is, this result suggests that TAM is not the only cell type which over-expresses IL-4 in the tumor microenvironment.

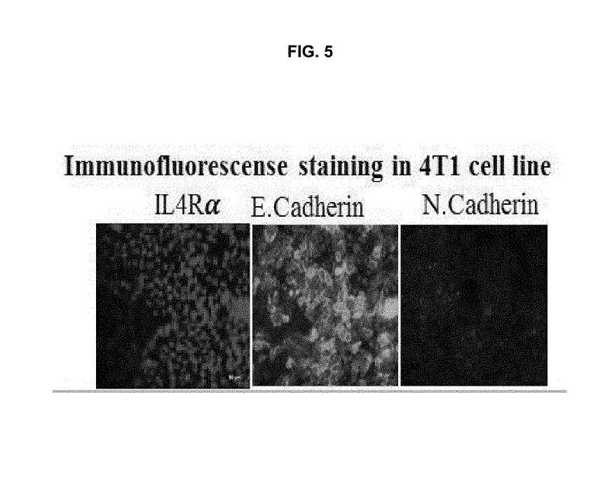

Therefore, in order to distinguish between TAM and 4T1 cells, the tumor tissues were stained with E-cadherin which is an epithelial cell marker of 4T1. Surprisingly, it was observed that the expression level of E-cadherin in 4T1 cells was extremely low as shown in the in vivo results of FIG. 4A, in contrast to the in vitro results of FIG. 5.

In order to confirm such results in more detail, the expression level of N-cadherin in 4T1 cells within the mouse tumor tissues was evaluated. N-cadherin is a marker known to be overexpressed in tumor cells which are in the state of epithelial mesenchymal transition. As shown in FIG. 4A, in contrast with the in vitro results, it was verified that N-cadherin was overexpressed in 4T1 cells within the tumor tissues of the wild-type mice. It is suggested that this difference between the in vivo and in vitro results may be associated with the in vivo epithelial mesenchymal transition of 4T1 cells, thereby increase in the expression of N-cadherin and IL-4 receptor of 4T1 cells, together with decrease in the expression of E-cadherin.

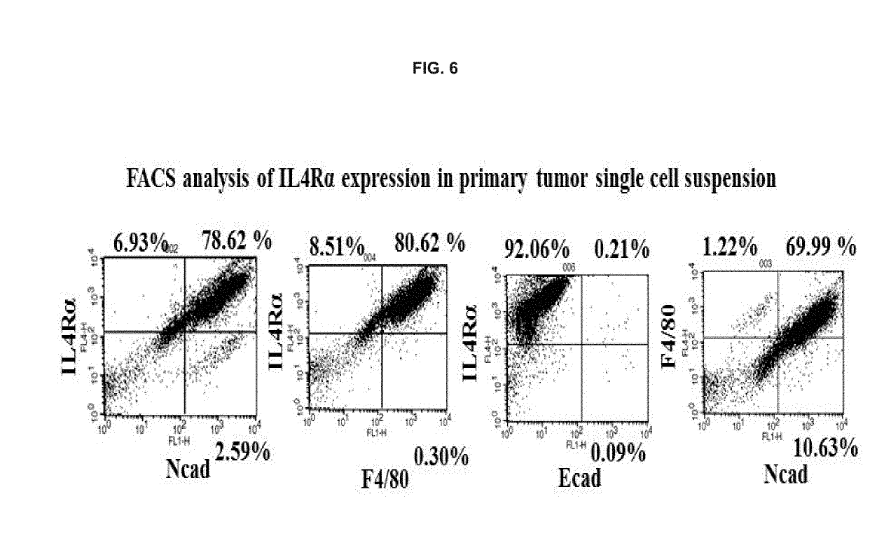

In order to further observe in more detail, 4T1 single cells within the tumor tissues of the mice were analyzed by flow cytometry (FACS). As shown in FIG. 6, it was observed that 4T1 cells expressing IL-4 receptors were lateralized with N-cadherin- and F4/80-expressing cells. This result suggests that the in vivo expression levels of both the epithelial mesenchymal transition marker and IL-4 receptor are increased in 4T1 cells which express small amount of IL-4 receptors in vitro

In other words, it can be understood that the tumor-associated macrophages (TAM) induce the expression of the IL-4 receptors in the 4T1 tumor cells.

### Example 4

Inducement of Epithelial Mesenchymal Transition and the Expression of IL-4 Receptors in 4T1 Cancer Cells by Tumor-Associated Macrophage (TAM) (In Vitro)

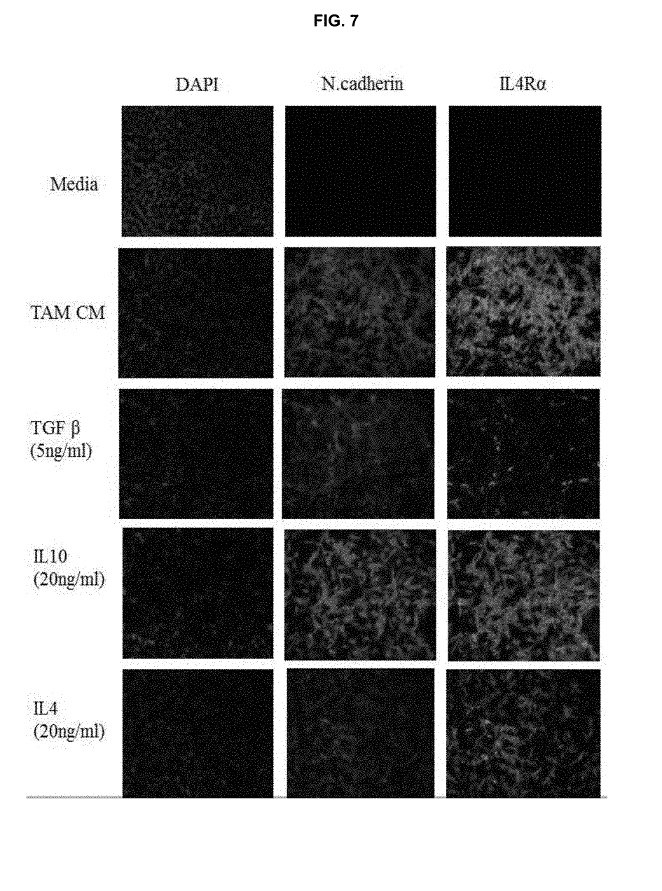

4T1 tumor cell lines were cultured in TAM condition medium, rmIL-10-containing medium, rmTGFβ-containing medium and rmIL-4-containing medium, respectively, in order to clearly resolve a point that the expression pattern of IL-4 receptors in 4T1 tumor cells is different between in vivo and in vitro and confirm that such a difference is closely related to tumor-associated macrophages (TAM). Results are shown in FIG. 7.

As shown in FIG. 7, the expression levels of a mesenchymal marker N-cadherin as well as IL-4 receptor were elevated in 4T1 cells which were cultured in both TAM condition medium and rmIL-10-containing medium, respectively.

Therefore, it was confirmed from the above results that TAM and IL-10 secreted therefrom are important factors in regulating the expression of IL-4 receptors in tumor cells, and in inducing epithelial mesenchymal transition

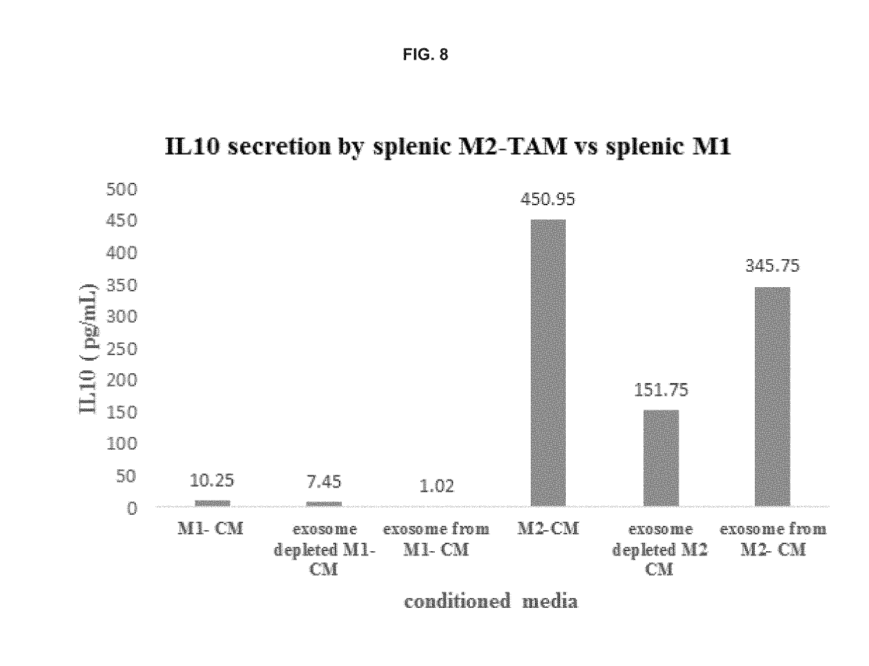

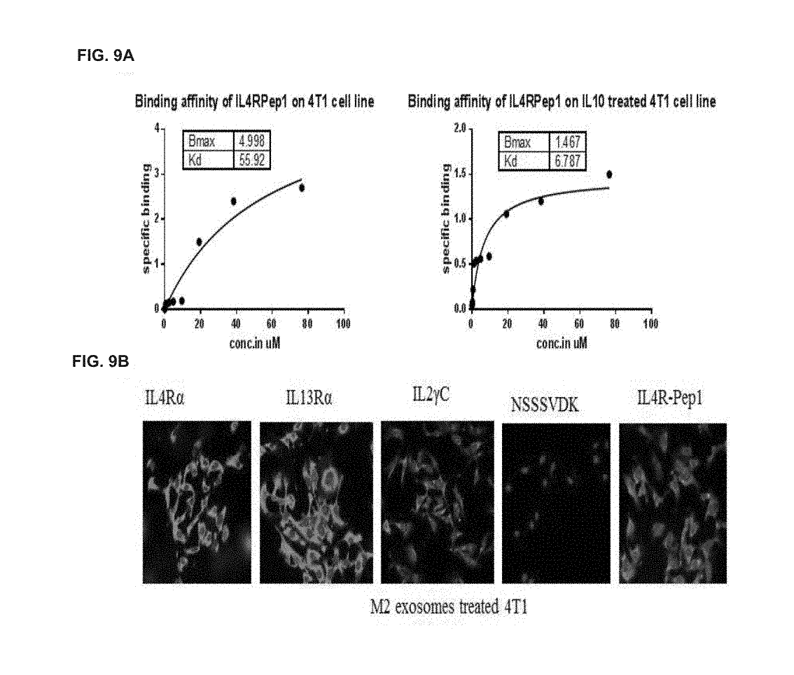

In order to demonstrate the above results more specifically, IL-10 production of M2 type macrophage TAM was compared with that of M1-type macrophage, and the expression level of IL-4 receptors and the binding pattern of the IL4RPep-1 peptides in the IL-10-treated 4T1 cells were confirmed by immunostaining. Results are shown in FIGS. 8, 9A & 9B.

As shown in FIG. 8, it was confirmed that M2 type macrophages secrete a large amount of IL-10 in the form of soluble cytokine or exosome, in contrast to almost no secretion of IL-10 by M1 type macrophages.

FIG. 9A shows that the binding affinity of the IL4RPep-1 peptides was significantly excellent in IL-10-treated 4T1 cells, compared with wild-type 4T1 cells. As shown in FIG. 9B, it was confirmed that, even in 4T1 cells treated with the exosomes of M2 type macrophages (TAM), a large amount of IL-4 receptors were expressed due to IL-10 secreted from TAM.

Based on the above results, it was verified that the expression of IL-4 receptors and epithelial mesenchymal transition in tumor cells are induced by tumor-associated macrophages (TAM), i.e. M2 type macrophages, more specifically, by IL-10 secreted by TAM, suggesting that TAM plays an important role in tumor progression into the metastasis phase.

### Example 5

Evaluation on Anticancer and Cancer Metastasis Inhibitory Activity of IL4RPep-1-KLA Fusion Polypeptide (SEQ ID NO: 3)

<5-1> In Vitro Cytotoxicity Test

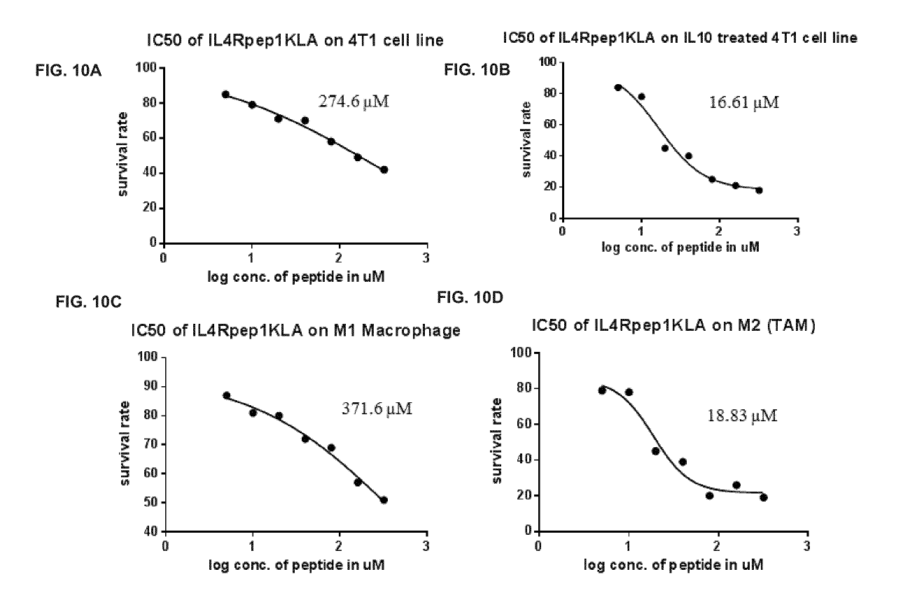

A cytotoxicity evaluation was performed on the fusion peptide (IL4RPep-1-KLA; SEQ ID NO: 3) in which the IL4RPep-1 peptide having the amino acid sequence of SEQ ID NO: 1 and the pro-apoptotic peptide (KLAKLAK)₂ are bonded. Results are shown in FIGS. 10A-10D.

As shown in FIGS. 10A-10D, the IL4RPep-1-KLA (SEQ ID NO: 3) exhibited an excellent cytotoxicity in the IL-10-treated 4T1 tumor cells and tumor-associated macrophages (TAM), i.e. M2 type macrophages. In contrast, the cytotoxicity of the IL4RPep-1-KLA fusion peptide (SEQ ID NO: 3) was insignificant in the wild-type 4T1 cells and the M1-type macrophages in which the expression of IL-4 receptor was seldom observed.

That is, the above results suggest that the IL4RPep-1-KLA fusion polypeptide (SEQ ID NO: 3) has excellent anticancer and cancer metastasis inhibitory effects by effectively targeting and killing IL-4-overexpressing tumor cells and M2 type macrophages, respectively.

<5-2> In Vivo Evaluation on Anti-Cancer and Cancer Metastasis Inhibitory Activity of IL4RPep-1-KLA Fusion Peptide (SEQ ID NO: 3), and Assessment of Co-Administration Efficacy

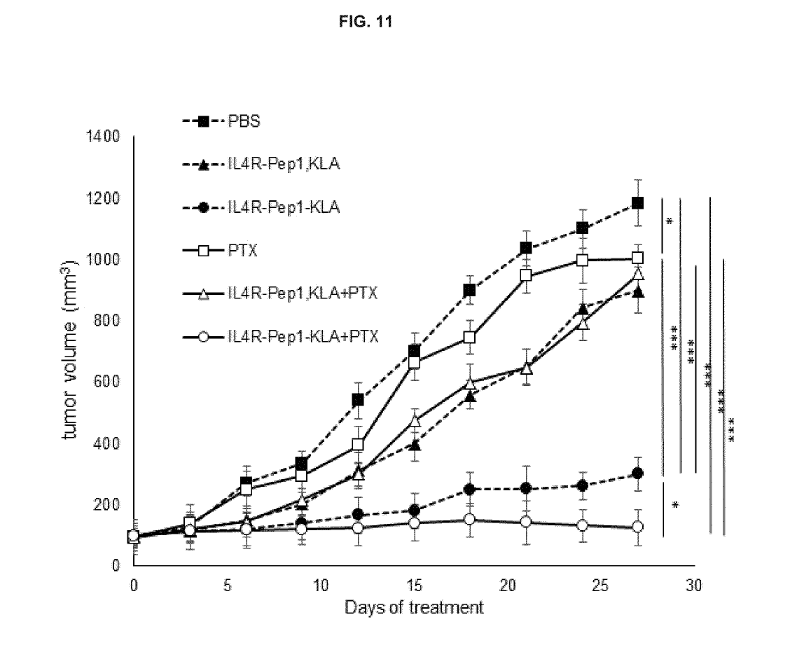

The anti-cancer and cancer metastasis inhibitory effects of the IL4RPep-1-KLA fusion polypeptide (SEQ ID NO: 3) were evaluated in Balb/c wild-type female mice transplanted with 4T1 cells. Results are shown in FIGS. 11, 12A, 12B, and 13.

As shown in FIG. 11, in the mice group which was administered with the IL4RPep-1-KLA fusion polypeptide, it was observed that the tumor growth was significantly suppressed from immediately after the administration up until the end of the experiment. Such a result was significantly superior to the result of the group in which the IL4RPep-1 peptide (SEQ ID NO: 1) and KLA (SEQ ID NO: 2) were administered individually. It is thus understood that the IL4RPep-1 peptide (SEQ ID NO: 1) effectively delivers the pro-apoptotic peptide KLA (SEQ ID NO: 2) to tumor cells by targeting tumor cells and tumor-associated macrophages.

On the other hand, when a widely used anti-cancer agent Paclitaxel (PTX) in an amount insufficient for anti-cancer effect as a monotherapy was administered in combination with the IL4RPep-1-KLA fusion polypeptide, its anti-cancer effect was significantly improved. This result suggests that the IL4RPep-1-KLA fusion polypeptide can be selected as a combination agent to maximize the therapeutic effect of conventional anti-cancer drugs.

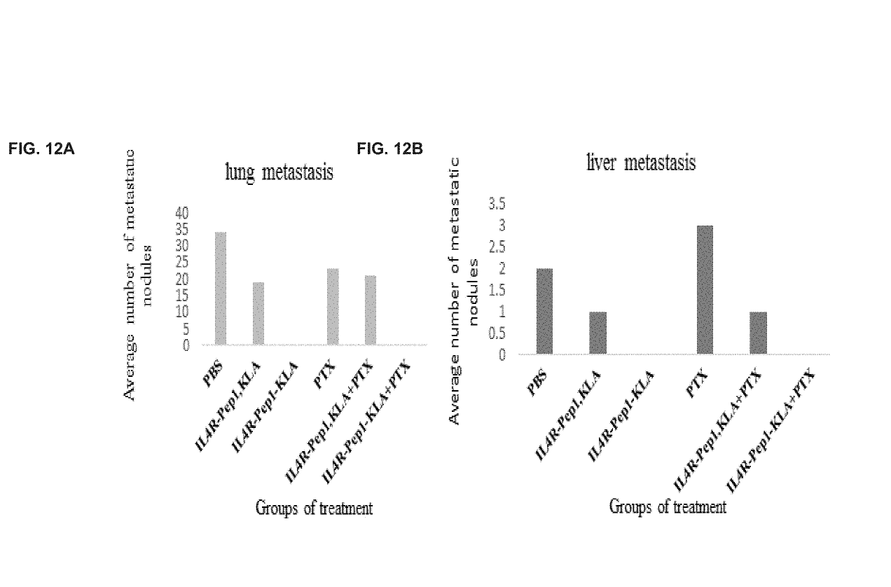

As shown in FIGS. 12A and 12B, after the end of the final administration, the liver and the lungs of each mouse were excised for observing the metastasis of cancer. As a result, the metastasis of cancer was not observed at all in the IL4RPep-1-KLA fusion peptide (SEQ ID NO: 3)-treated mouse group and IL4RPep-1-KLA fusion peptide (SEQ ID NO: 3)+paclitaxel (PTX)-treated mouse group, respectively, verifying that the IL4R-Pep-1-KLA fusion peptide is excellent in suppressing the metastasis of cancer and providing a combinatory effect with existing anti-cancer drugs.

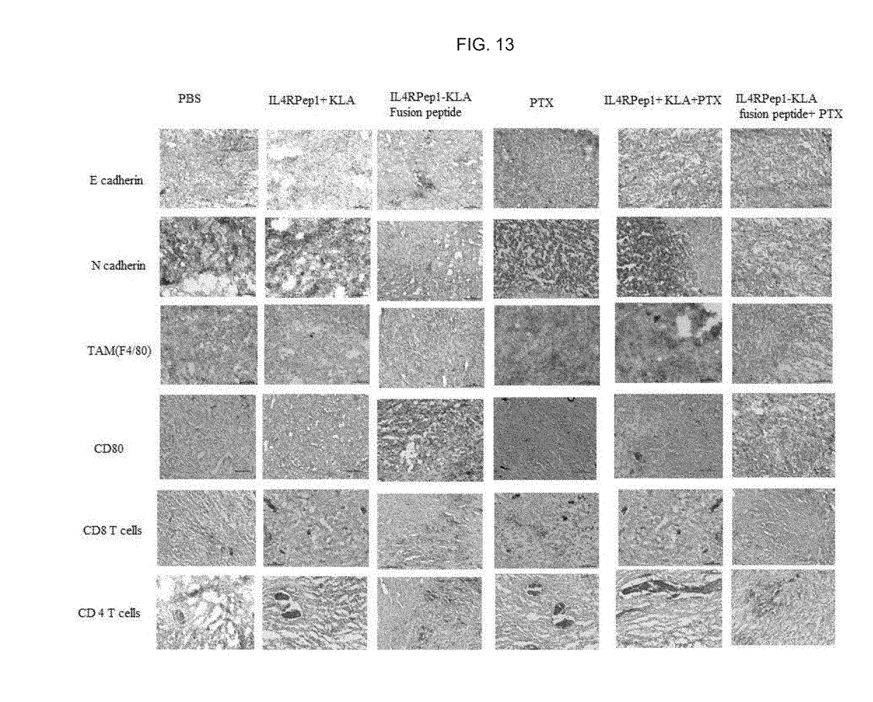

As shown in FIG. 13, after the completion of the final administration, frozen sections of the mouse tumor tissues were prepared and stained with the respective antibodies, followed by microscopic observation. As a result, in comparison with the control group, the IL4RPep-1-KLA fusion peptide (SEQ ID NO: 3)-treated mouse group and IL4RPep-1-KLA fusion peptide (SEQ ID NO: 3)+paclitaxel (PTX)-treated mouse group, respectively, showed reduced N-cadherins, reduced F4/80(+) tumor-associated macrophages, increased CD80(+) macrophages, increased CD8 (+) T cells, and decreased CD4(+) T cells.

### INDUSTRIAL APPLICABILITY

A pharmaceutical composition comprising the fusion polypeptide of the present invention as an active ingredient shows an excellent anti-cancer and cancer metastasis inhibitory effects by simultaneously targeting and killing tumor cells and tumor-associated macrophages and can be administered in combination with existing anti-cancer drugs to exert the anti-cancer and cancer metastasis inhibitory effects with reduced side effects of the conventional anticancer drugs, leading to its high industrial applicability.

What is claimed:

1. A pharmaceutical composition for treating cancer or suppressing cancer metastasis, the composition comprising as an active ingredient a fusion peptide in which a peptide having the amino acid sequence of SEQ ID NO: 1 and specifically targeting IL-4 receptor is linked to a pro-apoptotic peptide.

2. The composition of claim 1, wherein the composition simultaneously targets cancer cells and tumor-associated macrophage.

3. The composition of claim 1, wherein the fusion peptide is linked by a linker.

4. The composition of claim 1, wherein the pro-apoptotic peptide has the amino acid sequence of SEQ ID NO: 2.

5. The composition of claim 1, wherein the cancer is one in which IL-4 receptor is over-expressed.

6. The composition of claim 5, wherein the IL-4 receptor-overexpressed cancer is at least one selected from the group consisting of lung cancer, brain tumor, breast cancer, hepatic cancer, skin cancer, esophageal cancer, testicular cancer, renal cancer, colon cancer, rectal cancer, stomach cancer, kidney cancer, bladder cancer, ovarian cancer, cholangiocarcinoma, gallbladder cancer, uterine cancer, cervical cancer, prostate cancer, head and neck cancer, pancreatic cancer, and squamous cell carcinoma.

7. The composition of claim 1, wherein the composition is co-administered with an anti-cancer drug.

8. The composition of claim 7, wherein the anti-cancer drug is at least one selected from the group consisting of doxorubicin, paclitaxel, vincristine, daunorubicin, vinblastine, actinomycin-D, docetaxel, etoposide, teniposide, bisantrene, homo haeringtonine, imatinib (Gleevec; STI-571), cisplatin, 5-fluorouracil, adriamycin, methotrexate, busulfan, chlorambucil, cyclophosphamide, melphalan, nitrogen mustard, and nitrosourea.

9. A method for treating cancer or suppressing cancer metastasis, the method comprising administering to a subject in need thereof an effective amount of a fusion peptide in which a peptide having the amino acid sequence of SEQ ID NO: 1 and specifically targeting IL-4 receptor is linked to a pro-apoptotic peptide.

10. A fusion peptide in which a peptide having the amino acid sequence of SEQ ID NO: 1 and specifically targeting IL-4 receptor is linked to a pro-apoptotic peptide, for preparing an agent for treating cancer or suppressing cancer metastasis.
